(19) United States
(12) Patent Application Publication
(10) Pub. No.: US 2025/0319311 A1
(43) Pub. Date: Oct. 16, 2025

(54) HYBRID CONTROL USING VOLITIONAL CONTROL AND A CONTROL USER INTERFACE FOR FUNCTIONAL ELECTRICAL STIMULATION

(71) Applicant: Battelle Memorial Institute, Columbus, OH (US)

(72) Inventors: Gaurav Sharma, Lewis Center, OH (US); David A. Friedenberg, Columbus, OH (US); Douglas A. Thornton, Upper Arlington, OH (US); Justin C Sanchez, Vienna, VA (US)

(21) Appl. No.: 19/247,880

(22) Filed: Jun. 24, 2025

Related U.S. Application Data
(63) Continuation of application No. PCT/US2023/084557, filed on Dec. 18, 2023.
(60) Provisional application No. 63/435,411, filed on Dec. 27, 2022.

(51) Int. Cl. A61N 1/36; A61N 1/02; A61N 1/04; G16H 20/40; G16H 40/67

(52) U.S. Cl. CPC A61N 1/36031; A61N 1/025; A61N 1/0476; A61N 1/0484; A61N 1/36003; G16H 20/40; G16H 40/67

(57) ABSTRACT

A functional electrical stimulation (FES) system includes a stimulation garment with electrodes arranged to contact skin of an anatomical region worn on the anatomical region, an FES stimulator, an FES control user interface (UI) device configured to present an FES control UI, and a hardware processor programmed to: set the FES system in a user-selected operating mode based on user inputs from the FES control UI, determine an operating mode-specific FES stimulation based at least on the user-selected operating mode, and control the FES stimulator to apply the operating mode-specific FES stimulation to the anatomical region of the user via the electrodes.

This application is a continuation of PCT Application No. PCT/US2023/084557 filed Dec. 18, 2023 and published as WO2024/145051 A1, which claims the benefit of U.S. Provisional Patent Application Ser. No. 63/435,411 filed Dec. 27, 2022, which are each incorporated herein by reference in its entirety.

### BACKGROUND

The following relates to the neurological injury rehabilitation arts, to methods and apparatuses for aiding stroke recovery, methods and apparatuses for aiding spinal cord injury recovery, and to the like.

The following relates to improvements in functional electrical stimulation (FES) systems for purposes such as therapy, rehabilitation, assisting in activities of daily living (ADL), various combinations thereof, and/or so forth. Such systems may employ an electrical stimulation garment, which is designed to be worn on anatomy to receive the stimulation and has electrodes disposed on or in the garment arranged to contact the skin of the anatomy when the garment is worn on the anatomy. For example, an FES sleeve for applying FES to an arm and/or wrist and/or hand can be constructed as a compression sleeve of Lycra or another elastic material, with electrodes disposed on or woven into the inner surface of the sleeve so as to contact skin of the arm, wrist, and/or hand. Some nonlimiting illustrative examples of electrical stimulation garments are disclosed, for example, in Bouton et al., U.S. Pat. No. 9,884,178 issued Feb. 6, 2018 and Bouton et al., U.S. Pat. No. 9,884,179 issued Feb. 6, 2018, both of which are incorporated herein by reference in their entireties. Additional nonlimiting illustrative examples of electrical stimulation garments are disclosed, for example, in Blum et al., WO 2022/026821 A1 (PCT/US2021/043) published Feb. 3, 2022 and in U.S. Provisional Application No. 63/072,571 filed Aug. 31, 2020 titled “STRETCHABLE FABRIC SLEEVE FOR FUNCTIONAL ELECTRICAL STIMULATION AND/OR ELECTROMYOGRAPHY” and U.S. Provisional Application No. 63/058,776 filed Jul. 30, 2020 titled “STRETCHABLE FABRIC SLEEVE FOR FUNCTIONAL ELECTRICAL STIMULATION AND/OR ELECTROMYOGRAPHY”. Each of U.S. Pat. No. 9,884,178 issued Feb. 6, 2018, U.S. Pat. No. 9,884,179 issued Feb. 6, 2018, WO 2022/026821 A1 published Feb. 3, 2022, U.S. Provisional Application No. 63/072,571 filed Aug. 31, 2020, and U.S. Provisional Application No. 63/058,776 filed Jul. 30, 2020 is incorporated herein by reference in its entirety.

Control of an FES system may employ a brain-computer interface (BCI) which measures electrical activity in the motor cortex of the brain (or more generally brain electrical activity associated with motor cortical activity), and decodes volitional intent from measured brain electrical activity. The brain electrical activity serving as input to the BCI may be acquired via surface electrodes disposed on the scalp in electroencephalography (EEG), or via implanted intracortical electrodes (intracranial EEG or iEEG), or a Blackrock Utah microarray (available from Blackrock Neurotech, Salt Lake City, UT, USA), or a stent-electrode recording array (stentrode) implanted into a blood vessel in the brain, and/or so forth.

In another approach, electromyography (EMG) signals are measured using the electrodes of the electrical stimulation garment, and volitional intent is inferred from the measured EMG signals. Sharma et al., U.S. Pub. No. 2020/0406035 A1 titled “CONTROL OF FUNCTIONAL ELECTRICAL STIMULATION USING MOTOR UNIT ACTION POTENTIALS” discloses some approaches for EMG-based control. In one example, an electronic controller operatively connected with the electrodes is programmed to receive surface EMG signals via the electrodes of the garment, extract one or more motor unit (MU) action potentials from the surface EMG signals, identify an intended movement based at least on features representing the one or more extracted MU action potentials, and deliver FES effective to implement the intended movement via the electrodes of the wearable electrodes garment. The EMG control approach is premised on the patient's volitional intent generating neural signals to the muscles of the paralyzed body portion at sufficient strength to be detectable in the EMG signals, albeit at insufficient strength to stimulate (or fully stimulate) functional muscle contraction. The approach can be applicable to stroke patients, some spinal cord injury (SCI) patients, patients with motor impairment due to neurological disorders, or so forth.

Driving functional electrical stimulation by decoding volitional intent from input EEG, iEEG, or EMG signals is challenging for a number of reasons. The input signals can be noisy. The FES itself can introduce noise, especially EMG signal measurements are measured in between FES stimulation pulses. EEG signals may include brain electrical activity unrelated to motor cortical intent. iEEG can more precisely target the motor cortex, but at the cost of invasive implantation of intracortical electrodes; and the iEEG signal may still be contaminated with spurious brain electrical activity. FES control using EMG depends on assumptions about the extent of transfer of efferent motor cortical neural signals to the arm, wrist, hand, or other anatomy at which the EMG is measured. In practice, the extent of efferent motor cortical neural signal transfer to the anatomy can be limited, for example in the case of an SCI patient, or the efferent motor cortical neural signals may be partially misdirected in the case of a stroke patient. In practice, the BCI or EMG decoder typically employs an artificial neural network (ANN), support vector machine (SVM), or other machine learning (ML) algorithm to decode intent from the brain electrical activity or EMG. Due to interpatient variability, the ML component used in the intent decoding is individually trained to accommodate potentially substantial differences between patients.

On the other hand, control of an FES system by a BCI or EMG decoder has substantial advantages. Such control can readily be adapted or extended to implement new or different movements of the anatomy by training (or update training) of the ML component, and in some instances may constitute retraining potentially leading to partial recovery of functionality. Furthermore, controlling the FES by decoding the volitional intent of the patient provides substantial psychological benefits, as the patient is encouraged and empowered by directly controlling his or her own anatomy.

### BRIEF SUMMARY

In accordance with some illustrative embodiments disclosed herein, a functional electrical stimulation (FES) system comprises: a stimulation garment configured to be worn on an anatomical region of an associated user, the stimulation garment including electrodes arranged to contact skin of the anatomical region when the stimulation garment is worn on the anatomical region of the associated user; an FES stimulator operatively connected with the stimulation garment; an FES control user interface (UI) device configured to present an FES control UI; and a hardware processor. The hardware processor is programmed to: set the FES system in a user-selected operating mode based on user inputs received via the FES control UI, determine an operating mode-specific FES stimulation based on at least the user-selected operating mode, and control the FES stimulator to apply the operating mode-specific FES stimulation to the anatomical region of the associated user via the electrodes of the stimulation garment. In some embodiments, the FES system further comprises at least one neural signal amplifier configured to acquire neural signals indicative of motor cortex activity of the associated user, and the hardware processor is programmed to determine the operating mode-specific FES stimulation based on a volitional intent of the associated user and the user-selected operating mode including determining the volitional intent of the associated user by applying at least one machine learning (ML) component to the acquired neural signals.

In accordance with some illustrative embodiments disclosed herein, a non-transitory storage medium storing instructions readable and executable by a hardware processor to control an FES system that includes a stimulation garment configured to be worn on an anatomical region of an associated user, the stimulation garment including electrodes arranged to contact skin of the anatomical region when the stimulation garment is worn on the anatomical region of the associated user, an FES stimulator operatively connected with the stimulation garment, an FES control UI device configured to present an FES control UI, and at least one neural signal amplifier configured to acquire neural signals indicative of motor cortex activity of the associated user. The instructions are readable and executable by the hardware processor to control the FES system to perform operations including: setting the FES system in a user-selected operating mode based on user inputs received via the FES control UI; determining an operating mode-specific FES stimulation based on a volitional intent of the associated user and the user-selected operating mode including determining the volitional intent of the associated user by applying at least one ML component to the acquired neural signals; and controlling the FES stimulator to apply the operating mode-specific FES stimulation to the anatomical region of the associated user via the electrodes of the stimulation garment.

In accordance with some illustrative embodiments disclosed herein, a method of controlling an FES system is disclosed. The method includes: presenting an FES control UI on an FES control UI device and receiving user inputs via the FES control UI; setting the FES system in a user-selected operating mode based on user inputs received via the FES control UI; acquiring neural signals indicative of motor cortex activity; determining an operating mode-specific FES stimulation based on the neural signals and the user-selected operating mode; and applying the operating mode-specific FES stimulation to an anatomical region using electrodes of a stimulation garment. In some embodiments, the acquiring of neural signals includes acquiring at least one of electroencephalography (EEG) signals, intracranial EEG signals, and/or electromyography (EMG) signals.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically shows a functional electrical stimulation (FES) system combining volitional control with an FES control user interface (UI).

FIG. 2 diagrammatically shows an FES control method suitably performed by the hardware processor of the FES system of FIG. 1.

FIGS. 3-18 diagrammatically shows various menus of the control UI of the FES system of FIG. 1.

### DETAILED DESCRIPTION

To control functional electrical stimulation (FES), a control user interface can be implemented (for example as physical buttons or soft keys on a cellphone or tablet or other electronic device) to reliably evoke a desired movement. Alternatively, volitional intent can be inferred from physiological signals using machine learning (ML) algorithms, as in a brain-computer interface (BCI) or electromyography (EMG) decoder.

The former approach of using an FES control user interface (UI) allows for precise control but lacks the responsiveness and intuitiveness of physiologically decoded control. Button-based control can also be unsatisfying to the user, as he or she is not directly controlling the anatomy by volitional intent formed in the motor cortex. Still further, in the application space of rehabilitation, the decoupling of the intended movement with the movement required to activate the FES (e.g., pushing a button with an able left hand to cause a disabled right hand to grasp an object) reduces the potential for neuroplasticity and associated recovery of function.

The latter approach (e.g., BCI or EMG decoding) can be fast and intuitive, and can be satisfying for the user as the control is by direct volitional intent formed in the motor cortex (possibly as expressed by efferent motor cortical neural signals) but is susceptible to errors caused by variability in the sensing and decoding of the volitional intent from the brain neural activity or EMG. As previously noted, such decoding can be adversely affected by brain neural activity from regions of the brain other than the motor cortex, neural or electromyographic signals of the measured EMG that are unrelated to the volitional intent, and so forth, measurement error introduced by surface measurements in the case of EEG or EMG, and/or so forth.

In embodiments disclosed herein, combined systems are provided, which synergistically provide benefits of both systems while minimizing their weaknesses. Furthermore, using both volitional intent-based control (e.g. by decoding of EEG, iEEG, or EMG signals) and an FES control UI in combination adds additional depth to the quality and types of movements that can be controlled by the user. For example, the FES control UI can include a button (or alternative input, such as voice input, gaze tracking input, head movement input, sip-and-puff device input, or so forth) that is pressed (or otherwise input) to switch between different modes of physiological control, enabling the user to temporarily disable FES, switch the mapping between decoded movements and evoked movements, alternate between a decoded movement turning stimulation for the duration of decoded intent or turning stimulation on indefinitely (i.e., locked stimulation) until a STOP signal is received, and/or so forth. For certain movements such as picking up and placing an object, continuous stimulation control can be advantageous; whereas, for other movements such as carrying an object a locked mode can be desirable. (e.g., once the object is seized, that seizing action is locked during the carry of the object until the user wants to release the object).

By synergistically combining volitional control by (for example) decoding EEG, iEEG, and/or EMG signals with an FES control UI, the resulting FES control is intuitive and fast while also having a reduced error rate, and provides for refined control via different modalities, increased number of movements, context awareness, and/or so forth. In one illustrative example, the user employs the FES control UI to set the FES system for a particular task or context, and then EEG, iEEG, and/or EMG is decoded using a BCI and/or EMG decoder (typically employing a trained artificial neural network, ANN, or other ML component) to actively control the FES stimulation in a fast and intuitive manner. In some embodiments, the FES control UI allows for selecting a lock mode in which the stimulation is a sustained stimulation, and the EEG, iEEG, and/or EMG decoding then controls movement intent and timing. Termination of a locked movement can be triggered by further decoding of the EEG, iEEG, and/or EMG, or by another command (e.g. a STOP command) entered by the user via the FES control UI. In some embodiments, the FES control UI enables the user to place the FES system into a standby mode in which FES is not applied at all. The standby mode allows the user to temporarily disable the FES system and prevent false positive decodes from incorrectly evoking spurious movements when they are not intended. In some embodiments, if the EEG, iEEG, and/or EMG decoding is unable to decode more than a few different intended movements, the user could use the FES control UI to cycle between different movements (or contexts) and then use the intended movements that can be decoded to activate those different movements. Similarly, we could replace movements with other actions like controlling a smart home device or playing a game.

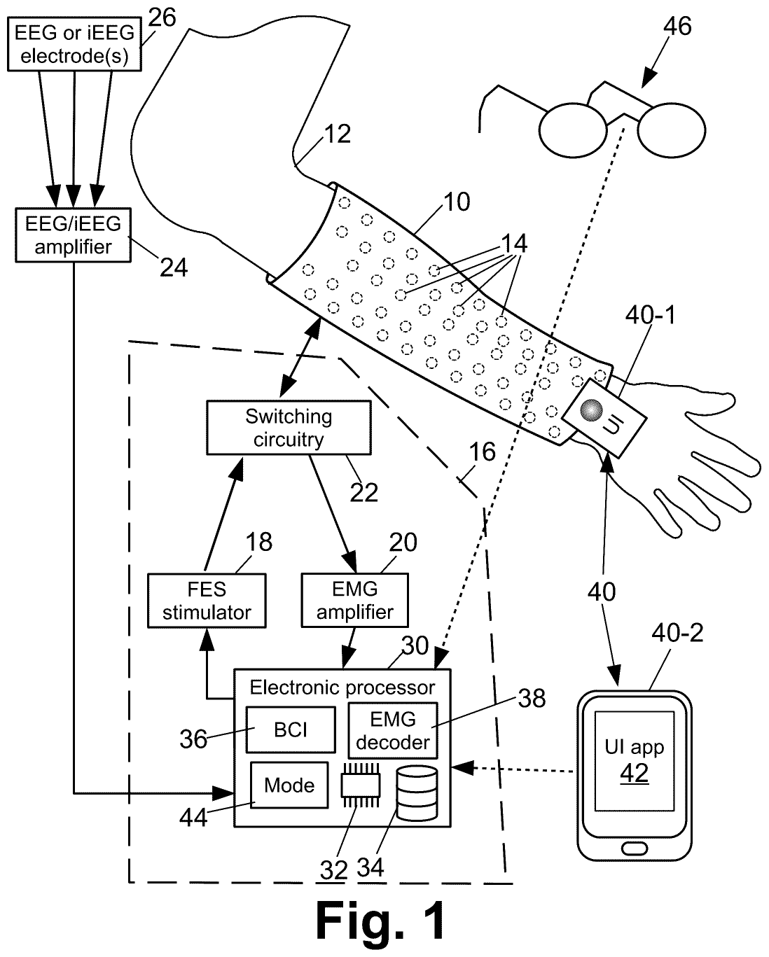

With reference to FIG. 1, a functional electrical stimulation (FES) system includes an electrical stimulation (FES) garment 10 that is wearable on an anatomical region 12, and includes a plurality of electrodes 14 contacting skin of the anatomical region 12 when the garment is worn on the anatomical region. The illustrative FES garment 10 is a sleeve 10 worn on an arm 12. In one embodiment, the sleeve 10 is made of Lycra or another elastic fabric so as to provide a compression fit to the anatomy 12—this compression fit presses the electrodes 14 (which are disposed on an inside surface of the sleeve 10 facing the skin) against the skin of the anatomy 12. More generally, the FES garment 10 may be made of a cloth, textile, polyester, or other material, and is sized and shaped to be worn on the anatomical region 12 to which FES is to be applied. The garment 10 may, for example, be a sleeve that is sized and shaped to be worn on an arm, a wrist, an ankle, an arm and a wrist, an arm and a wrist and a hand, a wrist and a hand, a leg, a leg and an ankle, or so forth. The sizing is suitably patient-specific to account for different anatomies of different patients, or the garment may be designed to be adjustable for differences between patients—for example, the sleeve could employ a wrap-around arrangement with Velcro to be adjustably wrapped around arms of different diameters, and/or made of Lycra or another elastic fabric that can fit a range of sizes. Suitable garments for a hand would include, for example, a glove or mitten.

The plurality of electrodes 14 are disposed on the inside of the garment 10 so as to contact the skin of the anatomical region 12. Note that FIG. 1 illustrates the garment 10 as transparent so as to reveal the underlying electrodes 14, but more typically the garment will be translucent or opaque. The electrodes 14 are connected by wires (possibly woven into the garment 10), circuitry of flexible printed circuit boards, and/or so forth (features not shown) to connect with electronics 16. The various components of the electronics 16 may be variously integrated with the FES garment 10, separate from the FES garment 10 and connected with the electrodes 14 by suitable electrical wires or cables or the like, or some combination thereof. Typically, the electrodes 14 are surface electrodes (e.g. electrogel discs). Embodying the electrodes 14 as needle electrodes or the like is also contemplated. In some embodiments the FES garment 10 is an elastic garment whose elasticity provides compressive force holding the electrodes 14 firmly against the skin of the wearer. The electrodes 14 are designed to provide good electrical contact with the skin of the anatomical region 12. For example, the electrodes 14 may be electrogel discs. Optionally, the garment may further include at least one Inertial Motion Unit (IMU) (not shown) such as an accelerometer, gyroscope, or the like, to provide information on the spatial orientation of the sleeve 10.

The electrodes 14 are configured to apply functional electrical stimulation (FES) pulses using an FES stimulator 18 which forms a portion of the electronics 16. By way of some non-limiting illustrative embodiments, some suitable FES pulse waveforms may include monophasic and biphasic pulses with a voltage between 80 to 300 Volts inclusive or higher. In one nonlimiting illustrative example, the FES pulse waveform is a monophasic pulse with a peak current of 0-20 mA which is modulated to vary strength of muscle contraction, frequency of 50 Hz, and a pulse width duration of 500 ms. Again, these are merely non-limiting illustrative examples.

With continuing reference to FIG. 1 the electrodes 14 may optionally also be used to measure electromyography (EMG) signals. The EMG signal measurements are potential difference measurements between pairs of electrodes 14 acquired using an EMG amplifier 20 which is also an optional component of the electronics 16. As recognized herein, the EMG signals may include efferent neural signals sent from the motor cortex of the brain of the patient to the anatomy 12; and/or the EMG signals may include electromyographic signals generated by muscles of the anatomy 12 in response to such efferent motor cortical neural signals. Hence, the EMG signals measured by the optional EMG amplifier 20 may encode volitional intent of the wearer (albeit possibly with some noise or transmission error, for example in the case of a stroke patient undergoing rehabilitation). The EMG potentials acquisition electronics may further include analog-to-digital (A/D) circuitry to convert the EMG signals to digital signal values. By way of nonlimiting illustrative example, the EMG amplifier 20 can be embodied as an Intan EMG amplifier (available from Intan Technologies, Los Angeles, California, USA). Because the EMG signals may be weak, it can be advantageous to integrate the EMG amplifier 20 with the sleeve 10 itself to minimize transmission distance from the electrodes 14 to the EMG amplifier 20. To enable switching between applying FES stimulation using the NMES stimulator 18 and receiving EMG measurements via the EMG amplifier 20, suitable switching circuitry 22 is provided, for example in various embodiments including solid state relays, high voltage field effect transistor (FET) components, and/or so forth, to enable the same set of electrodes 14 to switch between applying NMES stimulation and reading EMG signals. WO 2022/026821 A1 published Feb. 3, 2022 and U.S. Provisional Application No. 63/072,571 filed Aug. 31, 2020 and U.S. Provisional Application No. 63/058,776 filed Jul. 30, 2020, each of which is incorporated herein in its entirety, provides some suitable embodiments of the switching circuitry 22. It is noted that if the FES sleeve 10 is not also used to measure EMG, then the EMG amplifier 20 and switching circuitry 22 can be omitted.

With continuing reference to FIG. 1, in some embodiments the FES system includes an optional electroencephalography (EEG) or intracranial EEG (iEEG) amplifier 24 connected to receive neural signals indicative of motor cortex activity of the user, comprising EEG or iEEG signals received from EEG or iEEG electrodes 26, respectively. As previously noted, in some embodiments the optional EMG amplifier 20 is provided to receive neural signals indicative of motor cortex activity of the user, comprising EMG signals acquired from the anatomy 12. This latter approach relies on the user having sufficient neural connectivity from the user's brain to the anatomical region 12 so that the acquired EMG signals contain efferent neural signals indicative of motor cortex activity of the user. More generally, at least one neural signal amplifier 20, 24 is configured to acquire neural signals (e.g., EEG signals, iEEG signals, and/or EMG signals) indicative of motor cortex activity of the user. While the EMG amplifier 20 and EEG or iEEG amplifier 26 are provided as examples, an amplifier configured to acquire another type of neural signals indicative of motor cortex activity of the user is also contemplated. For example, the amplifier could be connected to acquire neural signals from an efferent nerve carrying neural signals from the motor cortex to the anatomical region 12 via an implanted electrode (for example, implanted in the neck or upper arm in the illustrative case where the anatomy 12 is an arm) accessing the efferent nerve.

The electronics 16 further include a hardware processor 30 for controlling the FES system. The illustrative hardware processor 30 comprises an electronic processor, such as a microprocessor or microcontroller 32, and a non-transitory storage medium 34. The microprocessor or microcontroller 32 may for example be programmed by software or firmware stored on the non-transitory storage medium 34 and readable and executable by the microprocessor or microcontroller 32 to perform the disclosed NMES functionality (and optional EMG measurement) in conjunction with the sleeve 10 and other components of the electronics 16 such as the NMES stimulator 18. The non-transitory storage medium may, for example, comprise a flash memory, solid-state drive (SSD), or other non-volatile electronic memory, although other types of media such as magnetic (e.g. a hard disk drive), optical (e.g. an optical disk) or so forth are additionally or alternatively contemplated. It is contemplated for the various components of the electronics 16 to be variously integrated with each other and/or variously integrated with the sleeve 10 (e.g., the EMG amplifier 20 could be embedded with or otherwise integrated with the stimulation sleeve 10 to reduce EMG signal transfer distance).

Among other provided functionality, the hardware processor 30 is programmed to determine the volitional intent of the user by applying at least one machine learning (ML) component to the neural signals acquired by the at least one neural signal amplifier 20, 24. In the illustrative example, the electronic processor is programmed to implement a brain-computer interface (BCI) 36 comprising an artificial neural network (ANN), support vector machine (SVM), or other ML component trained to determine the volitional intent of the user from the EEG or iEEG signals acquired by the EEG or iEEG amplifier 24. Additionally or alternatively, the electronic processor is programmed to implement an EMG decoder 38 comprising an ANN, SVM, or other ML component trained to determine the volitional intent of the user from the EMG signals acquired by the EMG amplifier 20. The trained ML component is suitably trained, for example, by a calibration session in which the user is instructed to form the intent to perform various movements and EEG, iEEG, and/or EMG signal data are recorded while the user is forming the intent. This provided labeled training data comprising the EEG, iEEG, and/or EMG signal data labeled with the intent that the user is instructed to form. The ML component is then trained on this training data, for example weights and activation functions of an ANN can be tuned to maximize fidelity of the volitional intent determined (i.e. output) the ANN with the labeled volitional intent. These are merely nonlimiting illustrative examples.

With continuing reference to FIG. 1, an FES control user input (UI) device 40 is also provided. FIG. 1 diagrammatically shows two suitable embodiments of the FES control UI device 40: a wrist-worn FES control UI device 40-1, or an FES control UI device 40-2 implemented as an application program (“app”) loaded on a cellphone or tablet computer. The FES control UI device 40 presents an FES control UI 42, which is diagrammatically indicated on the cellphone-or tablet-based FES control UI device 40-2 as an example, but could alternatively be presented via the wrist-worn FES control UI device 40-1. As will be described, control of the FES system is implemented by a synergistic combination of the volitional intent determined using the BCI 36 and/or EMG decoder 38 together with user inputs received via the FES control UI 42. In particular, in some embodiments the hardware processor 30 is programmed to set the FES system in a user-selected operating mode 44 based on user inputs received via the FES control UI 42. The user-selected operating mode 44 can be used in various ways to improve the performance of the FES control. In one approach, the user-selected operating mode 44 defines a context within which the FES system is used, and the BCI 36 and/or EMG decoder 38 applies ML component(s) specifically trained for that context. In another approach, the user-selected operating mode 44 is used to define constraints on the FES control. As an example of the latter approach, if the context is the user is performing a delicate task such as manipulating a toothbrush during brushing of teeth, then the FES can be constrained in intensity and/or duration to limit the force applied to the toothbrush.

Optionally, the FES system may include one or more auxiliary devices, such as illustrative eyeglasses 46 (or alternatively, a headset or the like) with gaze trackers to track the gaze of the user. In some operating modes such an auxiliary device 46 may be used to improve operation of the FES system.

In the FES system, volitional intent decoded from EEG, iEEG, EMG, other measured neural signals indicative of motor cortex activity of the user is combined with information such as a user-selected operating mode set based on user inputs received via the FES control UI 42. By combining these two systems into a hybrid FES controller the benefits of each approach are maintained while minimizing the disadvantages of each approach. Some illustrative use cases where the hybrid system provides improved functionality are described next.

In one example, the FES system may operate in either a user-selected continuous operating mode or a user-selected lock (i.e., sustained) operating mode. In a nonlimiting illustrative example, the BCI 36 and/or EMG decoder 38 decodes volitional movement intent continuously (e.g., 10 times per second as a nonlimiting example). Whenever movement intent is decoded from these signals the corresponding FES pattern is stimulated by the FES stimulator 18. This allows the user to control the precise timing of movement onset and termination. However, the continuous operating mode requires the user's continuous attention for the duration of the movement, and can be susceptible to small decoding errors leading to dropped objects or other mistakes. By contrast, in the user-selected lock (or sustained) operating mode, the hardware processor 30 determines a locked FES stimulation and controls the FES stimulator 18 to apply the locked FES stimulation continuously without update until a subsequent user input is received via the FES control UI 42 indicating the locked FES stimulation should be stopped. For example, in one nonlimiting example, the FES control UI 42 may include a button, softkey, or other input to allow the user to manually switch between continuous mode button control (i.e. hold the button down for the duration of the movement) or sustained control where the user presses once to initiate and then again to terminate, with the movement staying on indefinitely until the termination signal is received. Sustained movement is beneficial when, for example, holding an object (e.g. a coffee cup) where the user wants to maintain a grip without having to continuously think about it. With the lock mode, the user can use button presses (or other user input to the FES control UI 42) to switch between continuous or lock modes and, in the lock mode, use decoding to initiate or terminate movements with intuitive and precise timing.

In some embodiments, the FES control UI 42 may also provide for placing the FES system in a standby mode. There will be times when the user does not want to use the FES system, and the standby mode provides a convenient and reliable way to temporarily disable the system.

In some embodiments, the FES control UI 42 enables the user to set a context for operation of the FES system, and to switch between contexts as appropriate for the task at hand. In one example, the user can actively set the context via button presses or other user inputs to the FES control UI 42. The context can be leveraged in tuning the FES stimulation in various ways, such as using context-specific trained EEG, iEEG, and/or EMG decoders and/or imposing constraints on the FES stimulation.

In some embodiments, the FES control UI 42 can provide for movement substitution. A user with limited decodable physiological activity could cycle through different movements, using the buttons to select their intended movement and then initiate those movements by attempting a different movement that is easier to decode. In this way, the user can perform a movement that cannot be accurately sensed by EEG, iEEG, or EMG decoding by substituting a movement that can be accurately sensed as the trigger for the intended movement.

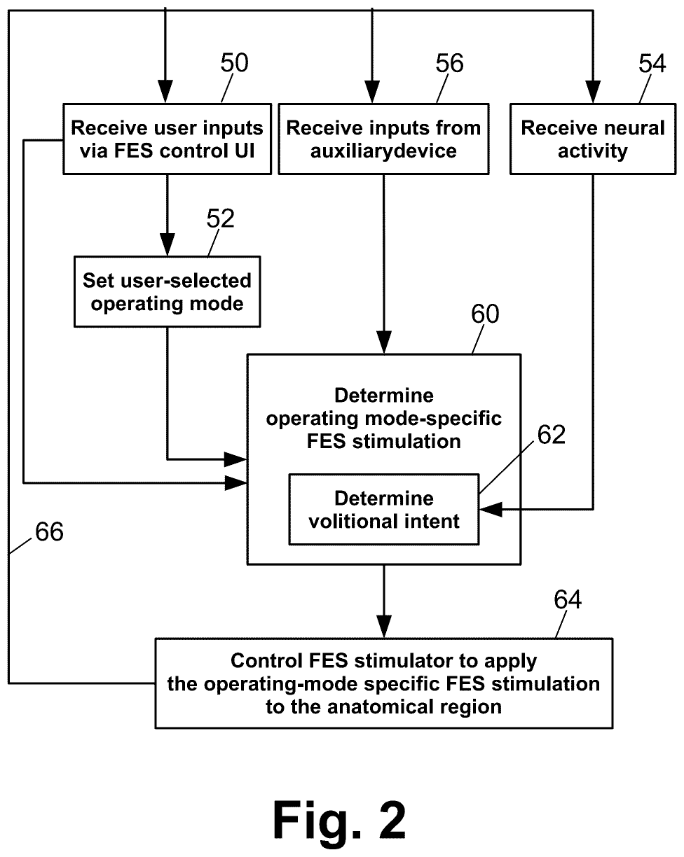

With reference now to FIG. 2, an illustrative FES control method suitably implemented by the FES system of FIG. 1 is shown. In an operation 50, user inputs are received via the FES control UI 42. In an operation 52 the user-selected operating mode 44 is set based on the user inputs received at the operation 50. For example, the user-selected operating mode could be a continuous mode, a lock mode, standby mode, a specific context mode, a guided task mode in which the FES control UI 42 will guide the user through a specific task, or so forth. It is contemplated in some embodiments for the user-selected operating mode to be a compound mode, such as a combination of a lock mode and a particular context mode. In such a case, the lock mode may be automatically chosen when that context is selected (e.g., selecting an object pick-up context may automatically switch the FES system to lock mode), or the user may in such embodiments select the lock mode (or continuous mode) independently of the selection of the context.

In an operation 54, neural activity (i.e., neural signals) indicative of motor cortex activity of the user are received. For example, this may be done by the EEG/iEEG amplifier 24 receiving EEG or iEEG signals from the motor cortex, or may be done by the EMG amplifier 20 receiving EMG signals from the electrodes 14 of the sleeve 10. Optionally, in an optional operation 56 further input may be received from one or more auxiliary devices, such as the illustrative eyeglasses 46 with gaze trackers.

In an operation 60, an operating mode-specific FES stimulation is determined based on a volitional intent of the user (determined in an operation 62) and the user-selected operating mode 44 set in the operation 52. The operation 62 determines the volitional intent of the user by applying at least one machine learning (ML) component to the neural signals acquired in the operation 54. In some user-selected operating modes, the operation 62 may employ context-specific ML components based on the context operating mode set in the operation 52. For example, the ML component for decoding volitional intent in the context of a precision activity such as brushing teeth may be differently optimized than the ML component for decoding volitional intent in the context of a more brute-force activity such as lifting a heavy object.

In some embodiments, the operation 60 may determine the mode-specific FES stimulation in which the intensity and/or duration of the operating-mode specific FES stimulation is constrained based on the user-selected operating mode. As one example, if the user-selected operating mode comprises a personal grooming context (e.g. suitable for brushing teeth, combing hair, shaving, or so forth) then the operation 60 may constrain the maximum FES stimulation intensity to be no larger than some maximum intensity to ensure the user cannot injure himself or herself by applying too much force to the toothbrush, comb, razor, or the like.

In some embodiments, the operation 60 may determine the mode-specific FES stimulation based on the received user inputs (other than or in addition to the user inputs that set the operating mode 44 in operation 52). For example, if the user-selected operating mode is a guided task then the user may input a START command to the FES control UI 42 to initiate FES stimulation, or a STOP or NEXT command to move to stop stimulation and/or to move to a next step in the guided task.

In an operation 64, the operating mode-specific FES stimulation determined in the operation 60 is executed by the hardware processor 30 controlling the FES stimulator 18 to apply the operating mode-specific FES stimulation to the anatomical region 12 of the user via the electrodes 14 of the stimulation garment 10. Thereafter, as indicated by flowback arrow 66 the process loops to enable the user to adjust the FES control by adjusting his or her volitional intent via operations 54 and 62, and/or by changing the operating mode via operations 50 and 52, and/or by providing other user inputs via operation 50.

In implementing the FES control UI 42, it may be desirable to limit the number of possible user input values needed to access all functionality provided by the FES control UI 42. For example, a quadriplegic may need to use a device such as a sip-and-puff device to operate the FES control UI 42. A sip-and-puff device typically comprises a head-mounted unit that places an air pressure and/or air flow sensor at the user's mouth, for example configured as a straw, wand, or the like. The user can provide inputs such as: value 1 corresponding to inhaling on the straw (i.e. a “sip”); or value 2 corresponding to exhaling into the straw (i.e. a “puff”). Additional values can be constructed by, for example, recognizing a set of two sips as a special value. However, it will be appreciated that the number of possible user input values that can be provided by a sip-and-puff device is low. As another example, a head-mounted accelerometer can be used as the input device for a quadriplegic. Again, the number of possible user input values is low in such a case, e.g. values corresponding to: head forward movement, head backward movement, and head-shake back-and-forth. More values can be constructed, for example by distinguishing between a head-forward movement and a head-far-forward movement, but the total number of possible input values is still limited. In some embodiments, the FES control UI 42 is configured to receive the user inputs of four or fewer possible user input values, thus accommodating limited-value user input devices such as a sip-and-puff device or a head-mounted accelerometer-based user input device.

On the other hand, in some other embodiments the FES control UI 42 may have a larger range of possible user input values. For example, if the FES control UI 42 is voice-controlled then the user can potentially provide many different user input values corresponding to a wide range of verbalized commands. Even in this case, however, it may be beneficial to limit the set of total possible user input values to a small number, as a small number of possible inputs is easier for the user to memorize and is easier for the user to learn the requisite muscle memory for making the inputs (this potentially being of particular importance, for example, in the case of some stroke patients).

To enable the FES control UI device 42 to provide a wide range of functionality with a limited set of possible user input values (e.g. four or fewer possible user input values in some nonlimiting illustrative embodiments), in some embodiments the FES control UI 42 is a menu-driven UI configured to receive the user inputs. By way of nonlimiting illustrative example, these inputs may, for example, comprise one or more of voice inputs, head movement inputs, sip-and-puff device inputs, mechanical input device actuations (e.g., mechanical buttons or keys, a mechanical slider switch, a joystick, and/or so forth), and/or softkey activations. The menu-driven UI may be hierarchical, and the user can navigate the menu-driven UI by a limited number of possible input values such as: input 1 to move a currently highlighted menu option down or to the right; input 2 to move the currently highlighted menu option up or to the left; input 3 to select the currently highlighted menu option; or input 4 to return to the main menu. This is merely a nonlimiting illustrative example.

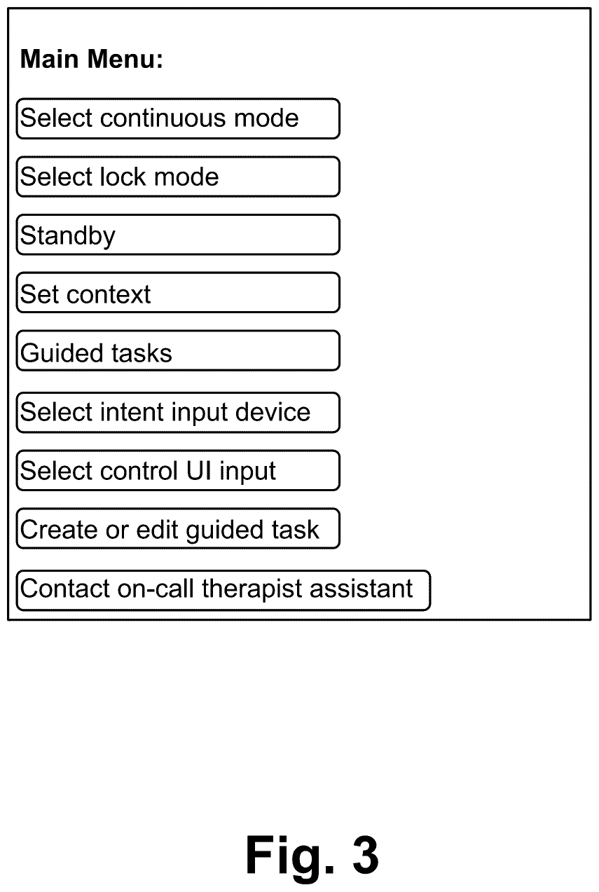

With reference now to FIGS. 3-18, a nonlimiting illustrative example of a menu-driven implementation of the FES control UI 42 is illustrated by way of presentation of various menus of one nonlimiting illustrative example of a hierarchical menu-driven FES control UI 42 that provides a wide range of functionality. In FIGS. 3-18, each menu option is diagrammatically indicated by a textual menu option label enclosed by a box. It will be appreciated that an actual implementation using a graphical user interface (GUI) approach may represent menu options in a wide range of ways, such as by underscored hyperlinks, text located in filled-in (possibly colored) boxes or the like, elements of a drop-down user dialog, checkbox dialogs, various combinations thereof, and/or so forth.

FIG. 3 presents the main menu, which provides various menu options. Some of these options set the user-selected operating mode (or lead to sub-menus for doing so). These include the following menu options: “Select continuous mode”, “Select lock mode”, “Standby”, “Set context”, and “Guided tasks”. Other menu options provide for user configuration of the FES control system (or lead to sub-menus for doing so). These include the following menu options: “Select intent input device”, “Select control UI input”, “Create or edit guided task”, and “Contact on-call therapist assistant”.

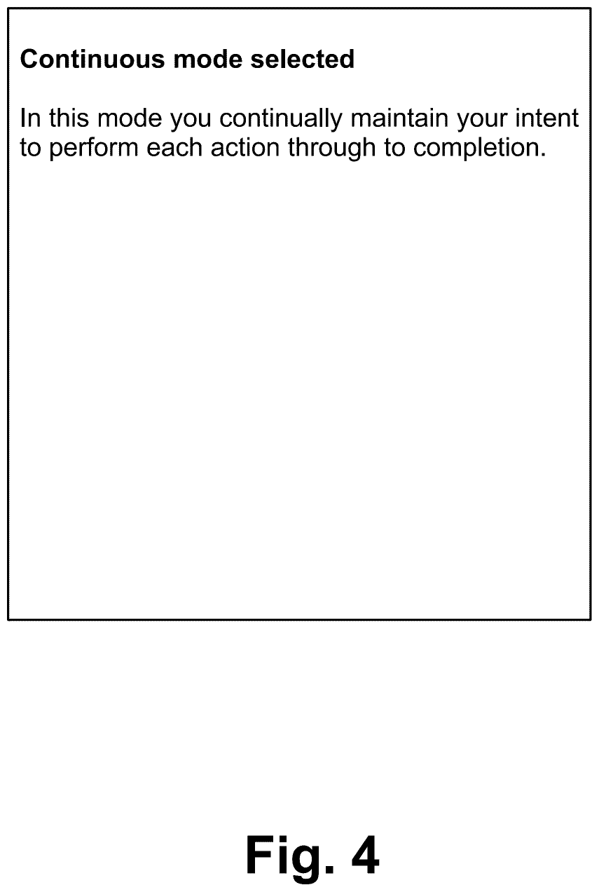

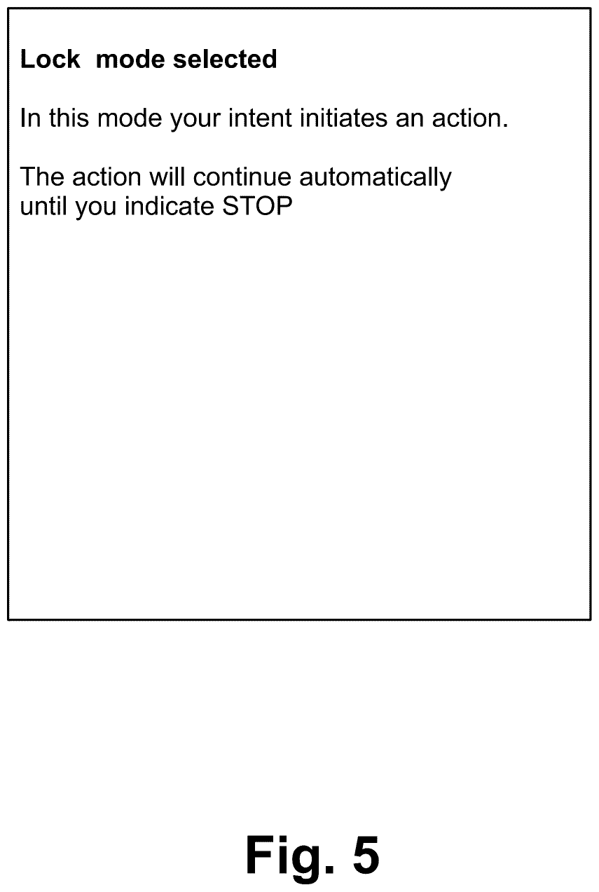

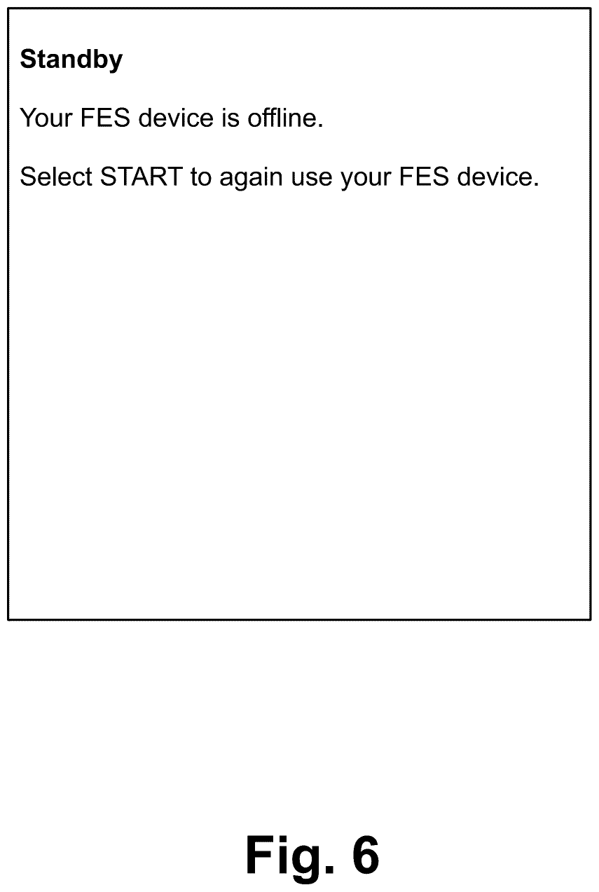

Selecting the “Select continuous mode” menu option of FIG. 3 brings up the display of FIG. 4 which explains that “in this mode you continually maintain your intent to perform each action through to completion.” Selecting the “Select lock mode” menu option of FIG. 3 brings up the display of FIG. 5 which explains that “in this mode your intent initiates an action. The action will continue automatically until you indicate STOP”. (In a variant embodiment, the action is initiated by a designated user input received by the FES control UI, and the text would then reflect that variant operation). Selecting the “Standby” menu option of FIG. 3 brings up the display of FIG. 6 which explains that: “Your FES device is offline. Select START to again use your FES device.” These menu options thus provide for general-purpose operation of the FES system, and for placing the FES system into standby.

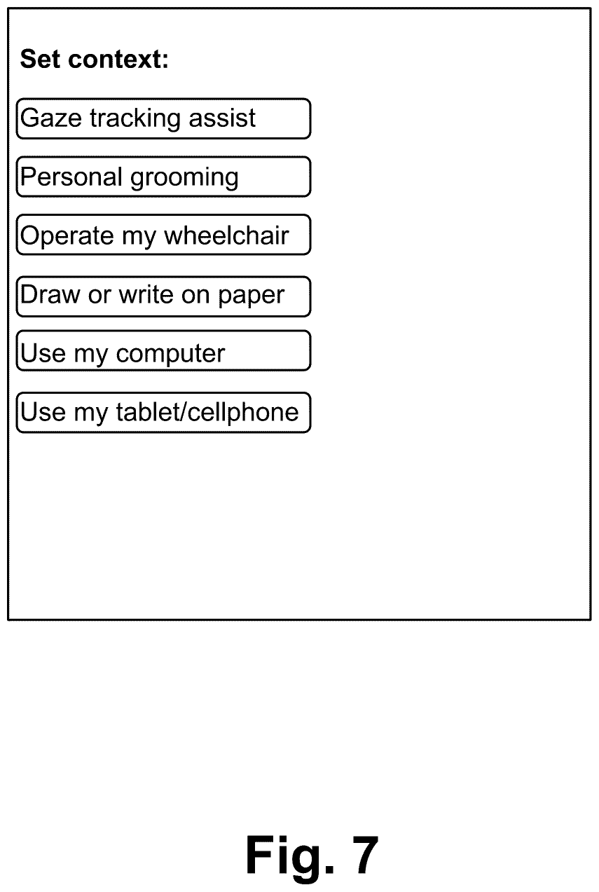

Selecting the “Set context” menu option of FIG. 3 brings up the “Set context” sub-menu shown in FIG. 7. In this nonlimiting illustrative example, contexts are provided including: “Gaze tracking assist”, “Personal grooming”, “Operate my wheelchair”, “Draw or write on paper”, “Use my computer”, and “Use my tablet/cellphone”. Selection of each of these options brings up a further submenu or display as described next.

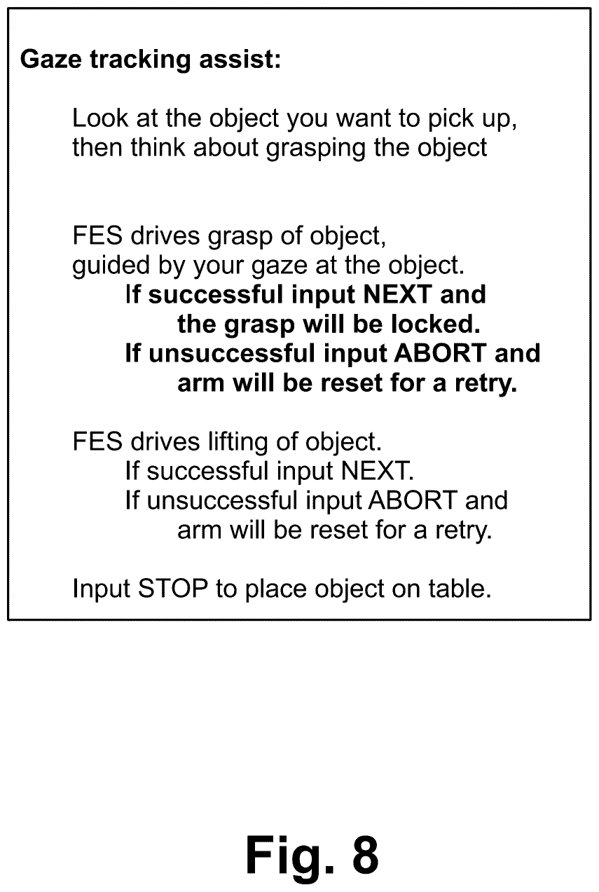

Selecting the “Gaze tracking assist” context brings up the display shown in FIG. 8 which provides step-by-step instructions for using the gaze tracking assist capability of the FES system (which utilizes the gaze trackers of the eyeglasses 46 of the FES system of FIG. 1, for example). The first step is explained as: “Look at the object you want to pick up, then think about grasping the object.” This is followed by: “FES drives grasp of object, guided by your gaze at the object.” This step may be implemented, for example, by having the eyeglasses 46 include a camera (or providing a camera at another suitable location) that captures video of the object and the FES sleeve 10 and performs image processing on video frames to provide video-feedback control of the applied FES stimulation to automatically guide the FES to move the hand to the object and grasp it. This is followed by: “If successful input NEXT and the grasp will be locked. If unsuccessful input ABORT and arm will be reset for a retry.” (In FIG. 8, this is the current step, as indicated by boldfacing of the text describing this step). This is followed by: “FES drives lifting of object. If successful input NEXT. If unsuccessful input ABORT and arm will be reset for a retry.” The final step is: “Input STOP to place object on table.”

In the example of FIG. 8, the gaze tracking assist mode leverages the gaze tracking eyeglasses 46 to identify the object to grasp, which can improve the accuracy of the FES system. Although not shown, other types of auxiliary devices can similarly improve the capability and/or accuracy of the FES system, such as: an auxiliary camera, an auxiliary electromagnetic (EM) tracking device, objects tagged with RF locator tags, and/or so forth. Such auxiliary devices can be provided with a corresponding assist context in the menu-based FES control UI 42 similarly to that shown for the gaze tracking assist of FIG. 8.

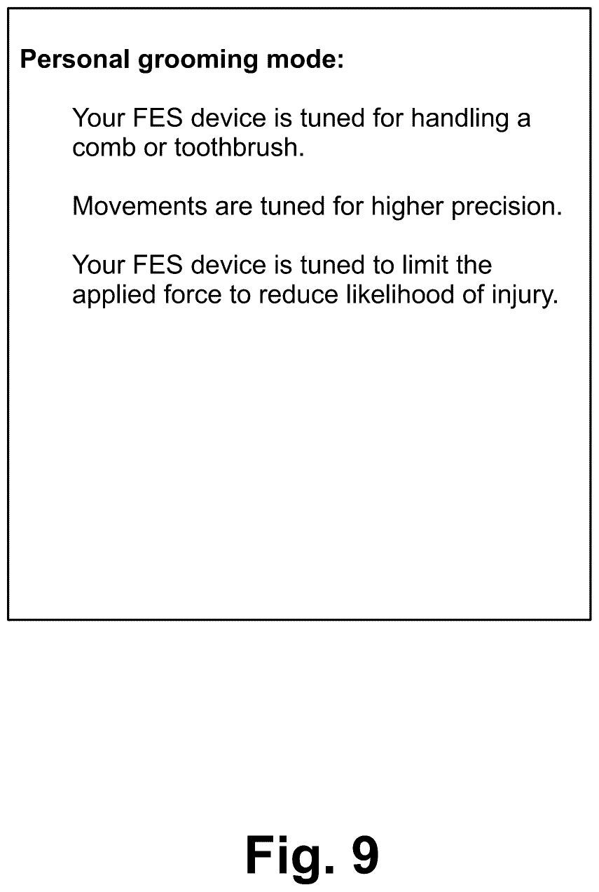

Returning to FIG. 7, selecting the “Personal grooming” context brings up the display shown in FIG. 9 which explains the operation of this context as follows: “Personal grooming mode: Your FES device is tuned for handling a comb or toothbrush. Movements are tuned for higher precision. Your FES device is tuned to limit the applied force to reduce likelihood of injury.” In implementation, these features can be provided by way of a ML component for the BCI 36 and/or the EMG decoder 38 that is trained for the context of personal grooming, and/or by imposing constraints on the intensity and/or duration of the FES stimulation to prevent the FES system from applying too much force to the comb, toothbrush or the like and/or to prevent the FES system from moving the comb, toothbrush or the like too far (since combing hair or brushing teeth typically involves short strokes of the comb or toothbrush). In this way, the FES control UI provides appropriate context to improve performance of the volitional control of the FES performed based on volitional intent decoded from the user's EEG, iEEG, and/or EMG.

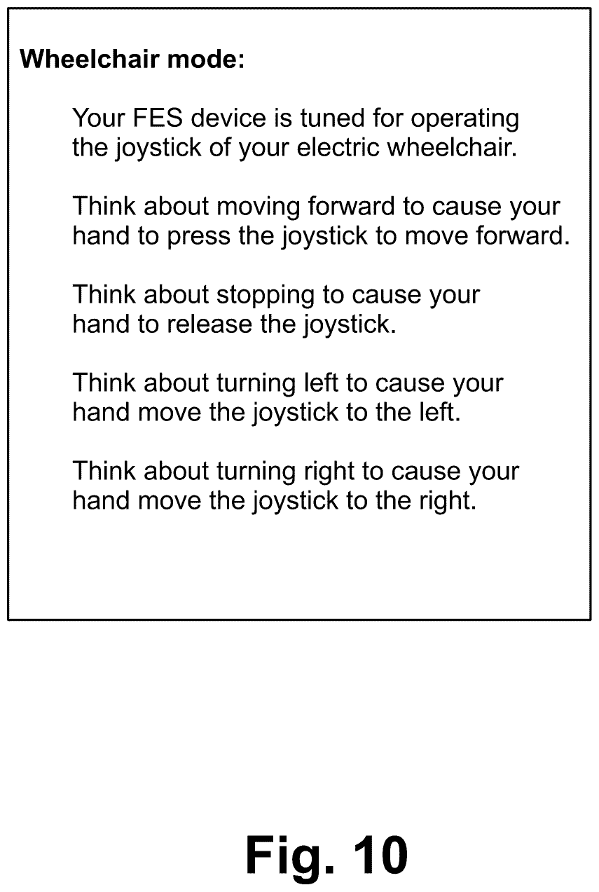

Returning to FIG. 7, selecting the “Operate my wheelchair” context brings up the display shown in FIG. 10 which explains the operation of this context as follows: “Your FES device is tuned for operating the joystick of your electric wheelchair. Think about moving forward to cause your hand to press the joystick to move forward. Think about stopping to cause your hand to release the joystick. Think about turning left to cause your hand move the joystick to the left. Think about turning right to cause your hand move the joystick to the right.” This user-selected operating mode context illustrates another optionally implemented feature of the hybrid volitional intent/FES control UI interface of the FES system, namely movement substitution. Here, the user's volitional intent is to move forward, and this volitional intent is translated into an FES stimulation that causes the user's hand to push the joystick forward thereby implementing the actual volitional intent of moving the wheelchair forward. This could be a useful substitution, since it may be easier for the user to formulate the intent to move forward which may be relatively easy to decode from EEG, iEEG, and/or EMG signals, and this is then translated to the intent to move the joystick forward, which might be a more difficult volitional intent to decode and moreover is not the end-result volitional intent of the user.

While this is one example, more generally a given context can instruct the user to intend a particular movement which will then be substituted by another movement. As another example, if the stimulation garment comprises leggings that provide FES stimulation to the leg muscles of the user, it may be easier for the user to intend to walk forward, and this singular intent is then translated into a sequence of FES stimulations to cause the muscles of the legs to operate in appropriate sequence to cause the user to actually walk forward.

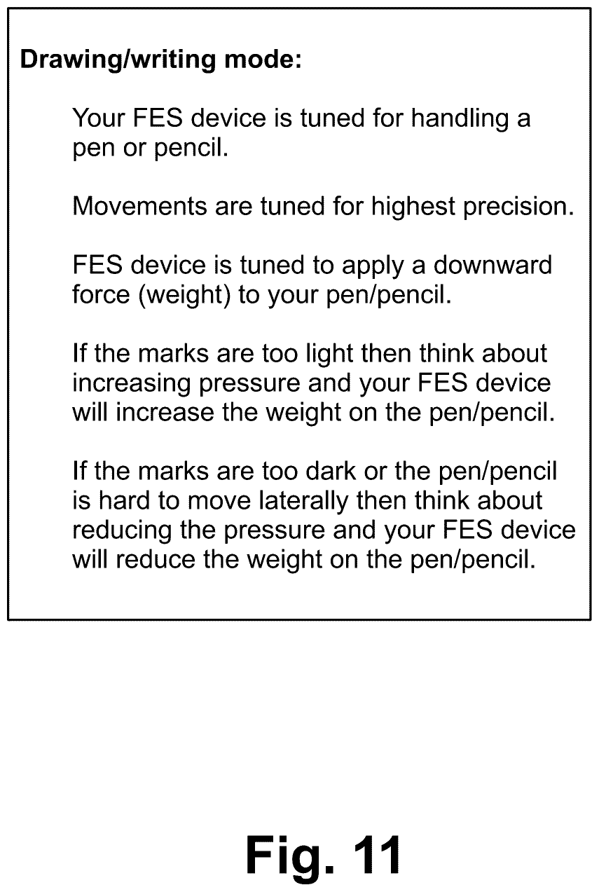

Returning to FIG. 7, selecting the “Draw or write on paper” context brings up the display shown in FIG. 11 which explains the operation of this context as follows: “Your FES device is tuned for handling a pen or pencil. Movements are tuned for highest precision. FES device is tuned to apply a downward force (weight) to your pen/pencil. If the marks are too light then think about increasing pressure and your FES device will increase the weight on the pen/pencil. If the marks are too dark or the pen/pencil is hard to move laterally then think about reducing the pressure and your FES device will reduce the weight on the pen/pencil.” This example demonstrates another advantage of the context-driven volitional FES control. Here, because the context is drawing or writing on paper, it is known that the FES stimulation should apply downward force on the pen or pencil being used to draw or write. Hence, the volitional intent (e.g., “darker” or “lighter”) is readily translated to FES stimulation to add or reduce downward force on the pen or pencil. In the absence of the known (user-selected) context, it would be easier for the FES system to misinterpret the decoded volitional intent as something incorrect or inappropriate for the task of writing or drawing on paper. Moreover, since the context of writing or drawing is known, the EEG, iEEG, and/or EMG decoding can optionally employ ML components for the decoding that were specifically trained for decoding volitional intent in the context of writing or drawing. For example, the context-specific ML component can be an ANN trained offline on training data limited to EEG, iEEG, and/or EMG neural signals recorded while the user was complying with requests related to writing or drawing and labeled with the corresponding requests, so that the trained ANN is specifically trained to decode intent in the context of writing or drawing. This, combined with the FES control UI 42 providing the user with the ability to select this particular context when appropriate substantially improves the decoding accuracy.

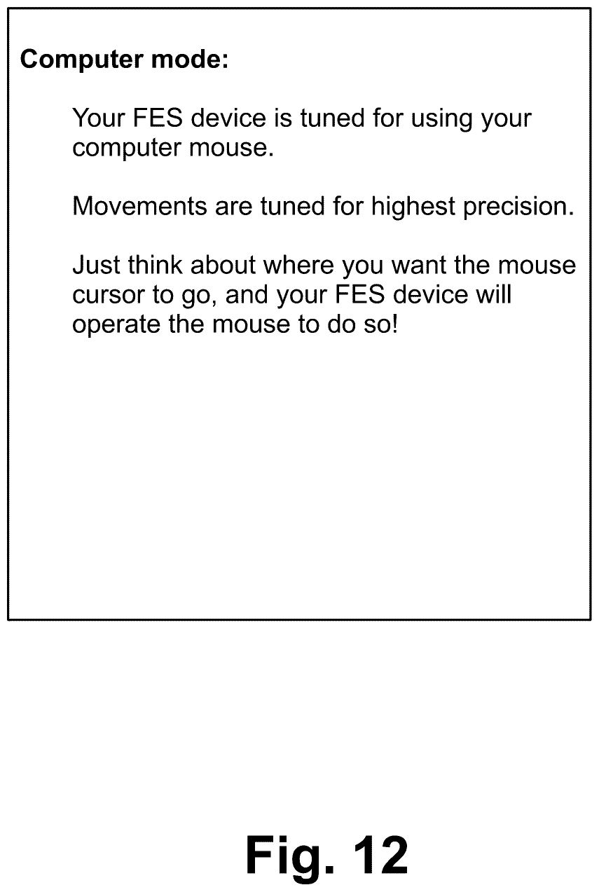

Returning to FIG. 7, selecting the “Use my computer” context brings up the display shown in FIG. 12 which explains the operation of this context as follows: “Your FES device is tuned for using your computer mouse. Movements are tuned for highest precision. Just think about where you want the mouse cursor to go, and your FES device will operate the mouse to do so!” Again, this employs movement substitution, where in this context the volitional intent to move the mouse point to a particular location on the screen is translated to an FES stimulation to move the mouse correspondingly. To implement this, referring briefly back to FIG. 2 the computer itself can be considered as an auxiliary device that in the operation 56 provides as input to the operation 60 the current mouse pointer location, so that the operation 60 can determine which way the pointer needs to go and hence which way to move the mouse. This context may also usefully employ constraint on the intensity and/or duration of the FES stimulation to ensure the movement of the mouse is small enough to avoid running the pointer into the edge of the screen. Also again, context-specific ML components can be used in the decoding, analogously to what was described above for the writing/drawing context.

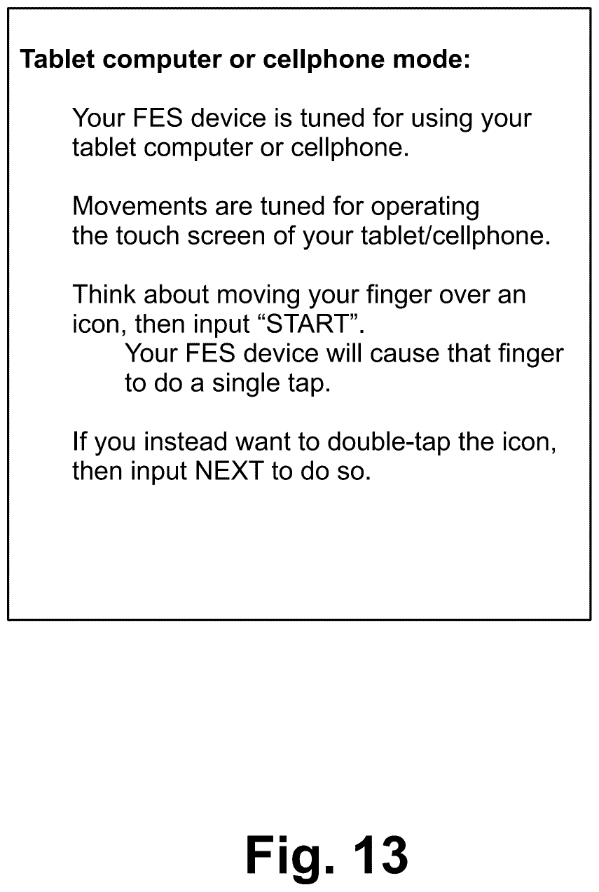

Returning to FIG. 7, selecting the “Use my tablet/cellphone” context brings up the display shown in FIG. 13 which explains the operation of this context as follows: “Your FES device is tuned for using your tablet computer or cellphone. Movements are tuned for operating the touch screen of your tablet/cellphone. Think about moving your finger over an icon, then input “START”. Your FES device will cause that finger to do a single tap. If you instead want to double-tap the icon, then input NEXT to do so.” Again, this context can leverage various context-specific FES control aspects such as employing context-specific ML components for decoding the volitional intent from the EEG, iEEG, and/or EMG, imposing suitable constraints on the intensity and/or duration of the FES stimulation, and (as in the computer operation embodiment previously described) receiving inputs from the tablet computer or cellphone which in this context serves as the auxiliary device providing the additional inputs for the operation 56 of FIG. 2.

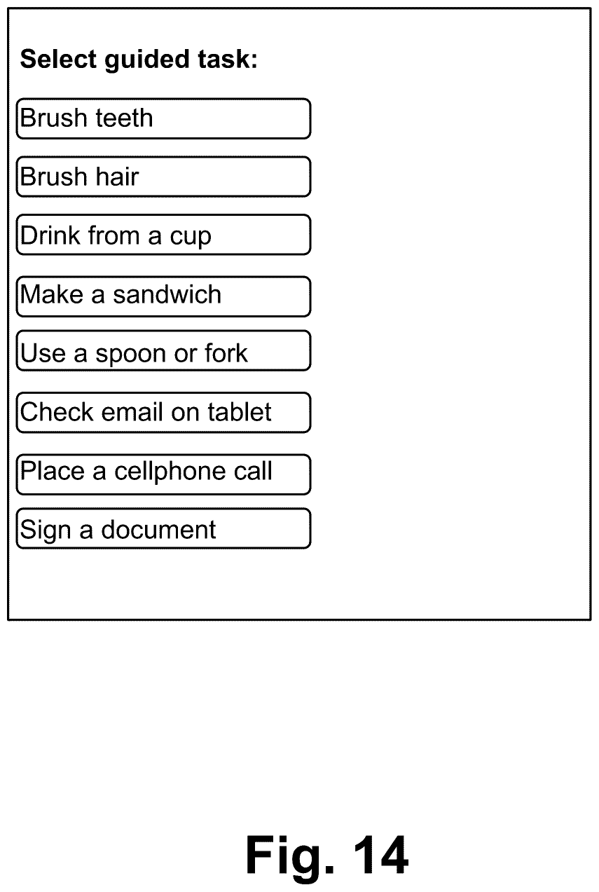

With reference back to the main menu of the FES control UI 42 shown in FIG. 3, selection of the main menu option “Guided tasks” brings up the guided tasks sub-menu shown in FIG. 14, which includes preprogrammed semi-automatic FES stimulation sequences for guided tasks including (in this nonlimiting illustrative example): brushing teeth, brushing hair, drinking from a cup, making a sandwich, using a spoon or fork, checking email on a tablet, placing a cellphone call, or signing a document. Implementation of each of these guided tasks is task-specific, but typically involves providing a display that indicates each step of the sequence and provides any instruction the user may need to perform (e.g.: “Input START to initiate ______ action”, “Input “NEXT” to move to the next action, et cetera) with the FES automatically providing FES stimulation to execute each step of the sequence. Some guided tasks may also leverage an auxiliary device to provide additional inputs for accurately providing FES stimulation support to perform the task, e.g. the “Drink from a cup” guided task may leverage the gaze trackers similarly to previously described with reference to FIG. 8 but in this case with the object known a priori to be a glass or the handle of a cup. Moreover, various of these guided tasks may leverage the lock mode (see FIG. 5 and related discussion) to implement a grasp-and-hold operation.

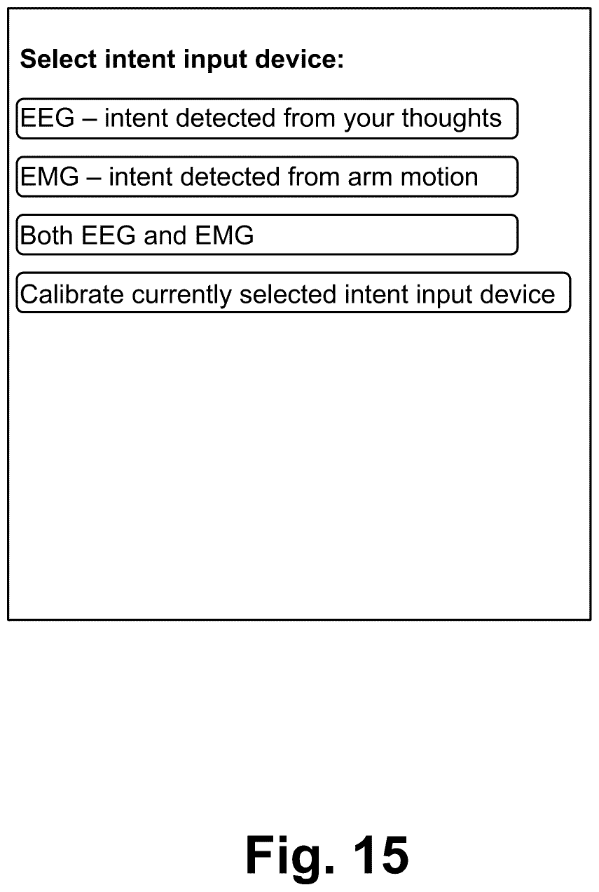

With reference back to the main menu of the FES control UI 42 shown in FIG. 3, selection of the main menu option “Select intent input device” brings up the sub-menu of FIG. 15 which provides menu options for selecting as the neural signal input for volitional intent decoding, including in this example: an EEG menu option; an EMG menu option; or a combined EEG and EMG menu option. In this last case, for example, the BCI 36 may decode the EEG and the EMG decoder may decode the EMG, and each decoder may provide a confidence or uncertainty value associated to the decoded intent. The operation 62 (see FIG. 2) then determines the volitional intent based on this information, e.g. of both EEG and EMG indicate the same volitional intent then that is the decoded intent; or if EEG and EMG indicate different volitional intents then the one with highest confidence (or lowest uncertainty) is suitably selected. The sub-menu of FIG. 15 also includes a “Calibrate currently selected intent input device” which can optionally bring up a training (or update training) module that provides supervised training in which the FES control UI 42 presents instructions to perform various movements while EEG and/or EMG (whichever is currently in use) is recorded, and this produced (additional) labeled training data for (update) training the ML component used in the decoding operation 62 of FIG. 2. Optionally, such training may also be specifically for a currently selected context (e.g., as selected via the sub-menu of FIG. 7) or for a particular guided task (e.g. as selected via the sub-menu of FIG. 14). The “select intent input device” sub-menu of FIG. 15 advantageously enables the FES system of FIG. 1 to be designed to work with EEG, EMG, or both (and, in variant embodiments, iEEG) without needing to redesign the FES system. To do so, the FES system suitably includes both the BCI 36 and the EMG decoder 38, with the appropriate module (or combination of modules) 36 and/or 38 used based on the selection the user makes via the sub-menu of FIG. 15.

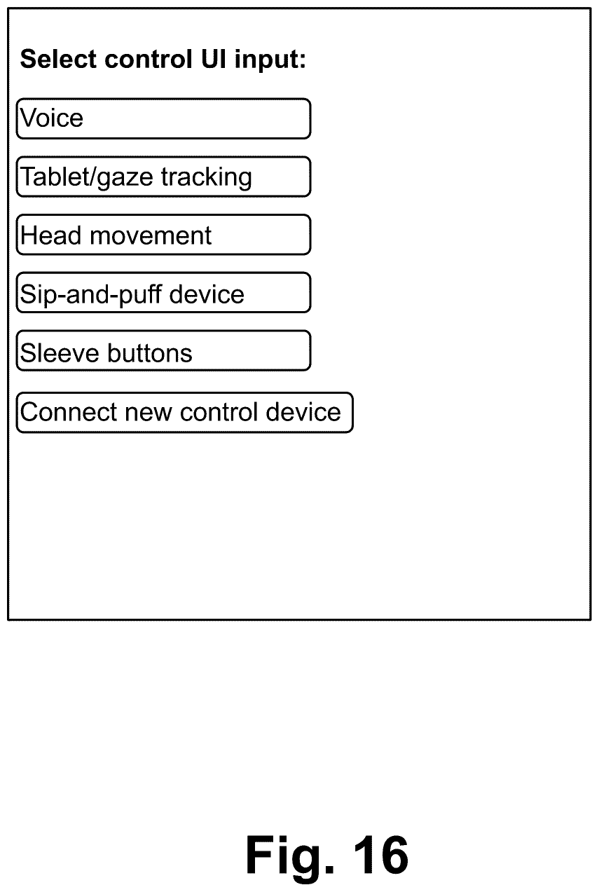

With reference back to the main menu of the FES control UI 42 shown in FIG. 3, selection of the main menu option “Select control UI device” brings up the sub-menu of FIG. 16 which provides menu options for selecting the input to the FES control UI device 40 for controlling the FES control UI 42. The illustrative sub-menu of FIG. 16 provides selectable inputs including: voice; tablet/gaze tracking; head movement; sip-and-puff device; and sleeve buttons. Voice control assumes the FES control UI device 40 includes a microphone (typically included in a cellphone as in the FES control device 40-2 of FIG. 1, and optionally included in the wrist-worn FES control UI device 40-1). Tablet/gaze tracking suitably leverages the gaze tracking provided by the auxiliary eyeglasses 46 to enable the user to select a menu option by gazing at it intently for a specified amount of time. Head movement input leverages an auxiliary device comprising an accelerometer mounted on the user's head to track head movements. Sip-and-puff device uses a sip-and-puff device (not shown) worn by the user. The sleeve button input uses mechanical buttons of the wrist-worn FES control UI device 40-1. These are merely illustrative examples. In some embodiments the sub-menu of FIG. 16 is exclusive so that the user can select only one UI input. In other embodiments, the sub-menu of FIG. 16 is not exclusive and the user can select two or more UI input devices (e.g., both voice input and sleeve buttons). It will again be appreciated that is aspect of the FES control UI 42 enables modularity, as the FES system of FIG. 1 can thereby be used with any of a set of different input devices (e.g., a microphone for voice input, head-mounted accelerometer, sip-and-puff device, et cetera) so long as a suitable device driver or application program interface (API) is provided for each available (or potentially available) UI device.

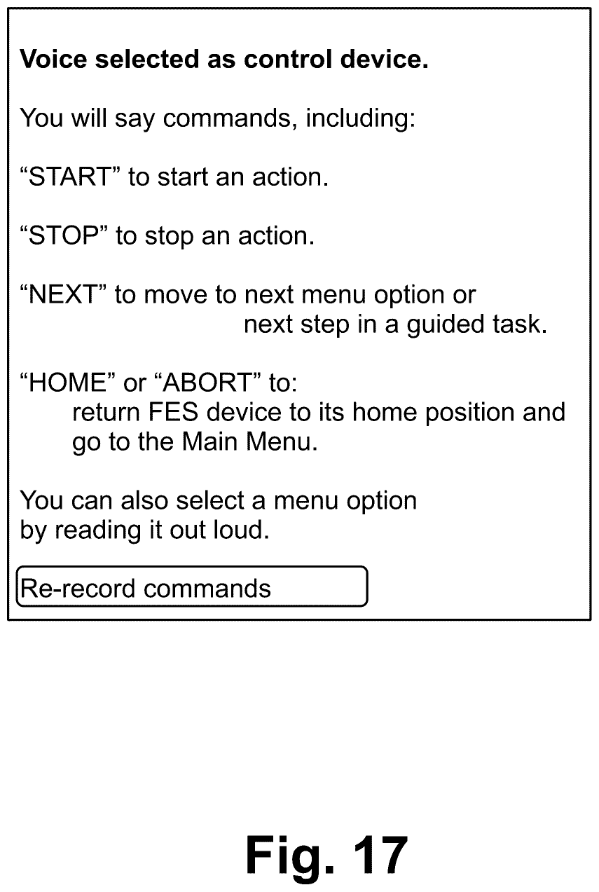

Optionally, selection of a given UI input via the sub-menu of FIG. 16 may bring up a further sub-menu for configurating that UI input. FIG. 17 illustrates an example where the user selects the “Voice” UI input menu option. This brings up the display shown in FIG. 17, which explains how to use voice UI control as follows: “You will say commands, including: ‘START’ to start an action. ‘STOP’ to stop an action. ‘NEXT’ to move to next menu option or next step in a guided task. ‘HOME’ or ‘ABORT’ to: return FES device to its home position and go to the Main Menu. You can also select a menu option by reading it out loud.” The display of FIG. 17 also provides a menu option labeled “Re-record commands”. This is provided because the voice input employs voice recognition, which can be individualistic. Hence, a given user may need to train the voice recognition by stating (for example) “START” several times so the voice recognition algorithm can learn to recognize how the individual normally says “START”.

Notably, there are only a small number of possible user input values: “START”, “STOP”, “NEXT”, and “HOME/ABORT”. Since voice input can theoretically handle any number of verbalized inputs, this limitation to a small number of inputs is not necessary for voice UI input. (This is captured in the final instruction shown in FIG. 17, i.e. “You can also select a menu option by reading it out loud.”) However, a small set of possible input values simplifies the learning process for the user and reduces likelihood of an erroneous input. Moreover, for more limited UI input devices the small number of possible inputs (e.g. four inputs in this example) are easily mapped to the limited physical inputs of such devices.

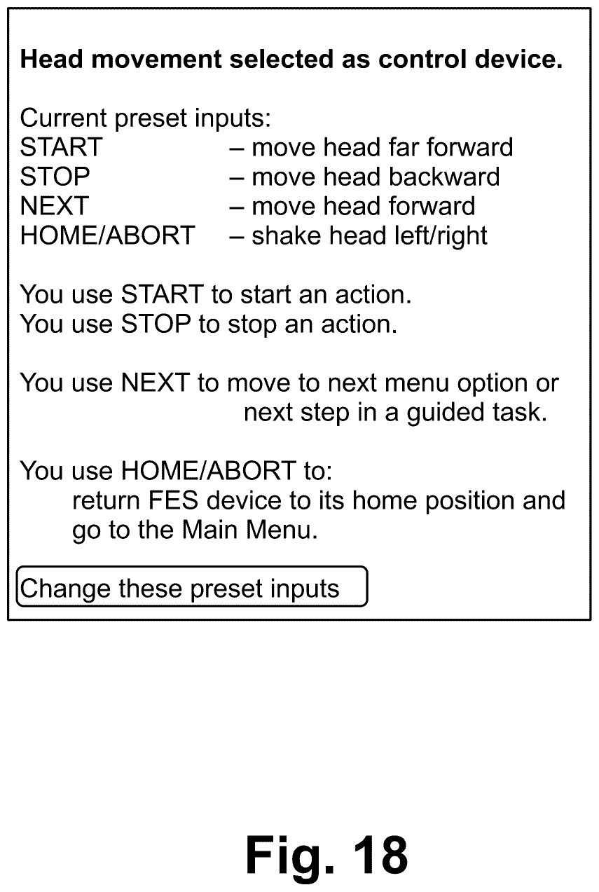

FIG. 18 illustrates this example for the limited physical inputs of a head movement UI device, providing the instructions: “Current preset inputs: START—move head far forward STOP—move head backward NEXT—move head forward HOME/ABORT—shake head left/right. You use START to start an action. You use STOP to stop an action. You use NEXT to move to next menu option or next step in a guided task. You use HOME/ABORT to: return FES device to its home position and go to the Main Menu.” Hence, a user who may, for example, be quadriplegic, can nonetheless navigate the complex menu system described with reference to FIGS. 3-18 with only four commands: START, STOP, NEXT, AND HOME/ABORT, using only head movements forward, backward, or shaking left/right. The display of FIG. 18 also provides a menu option labeled “Change these preset inputs” which brings up a further sub-menu or guided sequence of instructions (not shown) by which the user can reassign the mappings (e.g. reassign head backward to HOME/ABORT and shake back/forth to “STOP”, for example).

With reference back to FIG. 3, the user can navigate the main menu for example using the head movement input device by moving the head forward to move the currently highlighted menu option one step downward or moving the head backward to move the currently highlighted menu option one step upward, and using the head far-forward movement to select the currently highlighted menu option. At any time, the user can use the headshake back-forth as HOME/ABORT to go back to the main menu.

It is to be appreciated that the menu system depicted in FIGS. 3-18 is merely a nonlimiting illustrative example, and that numerous other menu architectures are contemplated, including these and/or other configuration options and user-selectable operating modes. In general, the FES control UI 42 enables the user to tune the FES system of FIG. 1 to a particular context, operating mode, guided task, or the like and thereby better tune and utilize volitional control based on EEG, iEEG, and/or EMG neural signals to provide a highly responsive FES system.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A functional electrical stimulation (FES) system comprising:
a stimulation garment configured to be worn on an anatomical region of an associated user, the stimulation garment including electrodes arranged to contact skin of the anatomical region when the stimulation garment is worn on the anatomical region of the associated user;
an FES stimulator operatively connected with the stimulation garment;
an FES control user interface (UI) device configured to present an FES control UI; and
a hardware processor programmed to:
set the FES system in a user-selected operating mode based on user inputs received via the FES control UI,
determine an operating mode-specific FES stimulation based at least on the user-selected operating mode, and
control the FES stimulator to apply the operating mode-specific FES stimulation to the anatomical region of the associated user via the electrodes of the stimulation garment.

2. The FES system of claim 1 further comprising:
at least one neural signal amplifier configured to acquire neural signals indicative of motor cortex activity of the associated user;
wherein the hardware processor is programmed to determine the operating mode-specific FES stimulation based on a volitional intent of the associated user and the user-selected operating mode including determining the volitional intent of the associated user by applying at least one machine learning (ML) component to the acquired neural signals.

3. The FES system of claim 2 wherein the at least one neural signal amplifier includes at least one of:
an electroencephalography (EEG) or intracranial EEG (iEEG) amplifier configured to acquire the neural signals comprising brain neural signals, and/or
an electromyography (EMG) amplifier configured to acquire the neural signals comprising EMG signals acquired using the electrodes of the stimulation sleeve.

4. The FES system of claim 2 wherein the hardware processor is programmed to:
set the FES system to the user-selected operating mode comprising a user-selected context based on the user inputs received via the FES control UI; and
the hardware processor is programmed to determine the volitional intent of the associated user by applying the at least one ML component comprising at least one context-specific ML component corresponding to the user-selected context to the acquired neural signals.

5. The FES system of claim 1 wherein the FES control UI comprises a menu-driven UI configured to receive the user inputs via comprising one or more of voice inputs, head movement inputs, sip-and-puff device inputs, mechanical input device actuations, and/or softkey activations.

6. The FES system of claim 5 wherein the menu-driven UI is configured to receive the user inputs of four or fewer possible user input values.

7. The FES system of claim 1 wherein the hardware processor is programmed to set the FES system in a user-selected operating mode that is selected from a set of available user-selectable operating modes including at least:
a continuous mode in which the hardware processor continuously updates the determination of the operating mode-specific FES stimulation;
a lock mode in which the hardware processor determines the operating-mode specific FES stimulation comprising a locked FES stimulation and controls the FES stimulator to apply the locked FES stimulation continuously without update until a subsequent user input is received via the FES control UI indicating the locked FES stimulation should be stopped.

8. The FES system of claim 7 wherein the set of available user-selectable operating modes further includes a standby mode in which the operating mode-specific FES stimulation is set to no stimulation.

9. The FES system of claim 1 wherein the hardware processor is programmed to:
set the FES system to the user-selected operating mode comprising a user-selected context based on the user inputs received via the FES control UI; and
the hardware processor is programmed to constrain an intensity and/or duration of the operating-mode specific FES stimulation based on the user-selected context.

10. The FES system of claim 1 wherein the hardware processor is programmed to:
set the FES system to the user-selected operating mode comprising a user-selected auxiliary device-assist mode based on the user inputs received via the FES control UI; and
the hardware processor is programmed to determine the operating mode-specific FES stimulation further based on an input received from an auxiliary device corresponding to the selected auxiliary device-assist mode.

11. The FES system of claim 10 wherein:
the selected auxiliary device-assist mode comprises a gaze tracker-assisted mode, and
the hardware processor is programmed to identify a target object based on a gaze of the associated user determined by the auxiliary device comprising a gaze tracker and to determine the operating mode-specific FES stimulation further based on the identified target object.

12. The FES system of claim 1 wherein the hardware processor is programmed to:
set the FES system to the user-selected operating mode comprising a user-selected guided task mode based on the user inputs received via the FES control UI;
present a sequence of actions for performing a selected task via the FES control UI when in the user-selected guided task mode; and
determine the operating mode-specific FES stimulation comprising a sequence of operating mode-specific FES stimulations corresponding to the sequence of actions presented by the FES control UI.

13. A non-transitory storage medium storing instructions readable and executable by an hardware processor to control a functional electrical stimulation (FES) system that includes a stimulation garment configured to be worn on an anatomical region of an associated user, the stimulation garment including electrodes arranged to contact skin of the anatomical region when the stimulation garment is worn on the anatomical region of the associated user, an FES stimulator operatively connected with the stimulation garment, an FES control user interface (UI) device configured to present an FES control UI, and at least one neural signal amplifier configured to acquire neural signals indicative of motor cortex activity of the associated user, the instructions being readable and executable by the hardware processor to control the FES system to perform operations including:
setting the FES system in a user-selected operating mode based on user inputs received via the FES control UI;
determining an operating mode-specific FES stimulation based on a volitional intent of the associated user and the user-selected operating mode including determining the volitional intent of the associated user by applying at least one machine learning (ML) component to the acquired neural signals; and
controlling the FES stimulator to apply the operating mode-specific FES stimulation to the anatomical region of the associated user via the electrodes of the stimulation garment.

14. The non-transitory storage medium of claim 13 wherein the instructions are readable and executable by the electronic processor to set the FES system into at least:
a continuous mode in which the determination of the operating mode-specific FES stimulation is continuously updated; and
a lock mode in which the operating-mode specific FES stimulation comprising a locked FES stimulation is determined and maintained without update until a subsequent user input is received via the FES control UI indicating the locked FES stimulation should be stopped.

15. The non-transitory storage medium of claim 13 wherein the instructions are readable and executable by the electronic processor to set the FES system into a standby mode in which the operating mode-specific FES stimulation is set to no stimulation.

16. The non-transitory storage medium of claim 13 wherein the instructions are readable and executable by the electronic processor to:
set the FES system into the user-selected operating mode comprising a user-selected context based on the user inputs received via the FES control UI; and
determine the volitional intent of the associated user by applying the at least one ML component comprising at least one context-specific ML component corresponding to the user-selected context to the acquired neural signals.

17. The non-transitory storage medium of claim 13 wherein the instructions are readable and executable by the electronic processor to:
set the FES system into the user-selected operating mode comprising a user-selected context based on the user inputs received via the FES control UI; and
constrain an intensity and/or duration of the operating-mode specific FES stimulation based on the user-selected context.

18. The non-transitory storage medium of claim 13 wherein the instructions are readable and executable by the electronic processor to:
set the FES system to the user-selected operating mode comprising a user-selected auxiliary device-assist mode based on the user inputs received via the FES control UI; and
determine the operating mode-specific FES stimulation further based on an input received from an auxiliary device corresponding to the selected auxiliary device-assist mode.

19. The non-transitory storage medium of claim 13 wherein the instructions are readable and executable by the electronic processor to:
set the FES system to the user-selected operating mode comprising a user-selected guided task mode based on the user inputs received via the FES control UI;
present a sequence of actions for performing a selected task on the FES control UI when in the user-selected guided task mode; and
determine the operating mode-specific FES stimulation comprising a sequence of operating mode-specific FES stimulations corresponding to the sequence of actions presented by the FES control UI.

20. A method of controlling a functional electrical stimulation (FES) system, the method including:
presenting an FES control user interface (UI) on an FES control UI device and receiving user inputs via the FES control UI;
setting the FES system in a user-selected operating mode based on user inputs received via the FES control UI;
acquiring neural signals indicative of motor cortex activity;
determining an operating mode-specific FES stimulation based on the neural signals and the user-selected operating mode; and
applying the operating mode-specific FES stimulation to an anatomical region using electrodes of a stimulation garment.
